(19) United States
(12) Patent Application Publication
(10) Pub. No.: US 2010/0022816 A1
(43) Pub. Date: Jan. 28, 2010

(54) Dehydrogenation of Methylbutenes to Isoprene

(75) Inventor: James T. Merrill, Morgan, TX (US)

Correspondence Address: FINA TECHNOLOGY INC, PO BOX 674412, HOUSTON, TX, 77267-4412, US

(73) Assignee: Fina Technology, Inc., Houston, TX (US)

(21) Appl. No.: 12/177,740

(22) Filed: Jul. 22, 2008

(51) Int. Cl. C07C 5/333; C07C 5/327

(52) U.S. Cl. 585/661; 585/654

(57) ABSTRACT

A method for the dehydrogenation of isoamylene to isoprene at pressures less than atmospheric utilizing a dehydrogenation catalyst is disclosed. Embodiments involve operating the dehydrogenation reactor at a pressure of 1,000 mbar or less.

### CROSS-REFERENCE TO RELATED APPLICATIONS

Not applicable.

### FIELD

The present invention generally relates to the production of isoprene.

### BACKGROUND

Isoprene is the common name for the chemical compound known as 2-methyl-buta-1,3-diene, that is found in natural rubber. Isoprene is used as a starting material for the production of synthetic versions of natural rubber including polyisoprene and various isoprene-based rubbery copolymers such as styrene-butadiene type copolymers. Isoprene can be produced using a variety of processes. These can include, for example, byproducts of various refining operations such as the thermal cracking of naphtha or oil; the dehydrogenation of isoamylene compounds; the pyrolysis of allylic esters; and the decomposition of dioxane.

Isoamylene may be a component of a C₅ refinery stream. The isoamylene portion of such a stream will typically contain at least two isoamylene monomers, i.e., 2-methyl-2-butene and 2-methyl-1-butene, often in a weight ratio of about 1:1 to about 10:1, and most often between 1:1 and 5:1, respectively. A third monomer, 3-methyl-1-butene may also be present but is typically in much lower amounts than the other two monomers. Isoprene can be produced by the catalytic dehydrogenation reaction of isoamylene in the presence of oxygen. The oxygen is typically provided by adding steam to the reaction zone. Isoamylene is also commercially available from chemical manufacturers such as Ineos, which markets an isoamylene product comprising approximately 85 wt % 2-methyl-2-butene and 15 wt % 2-methyl-1-butene.

It may be desirable to utilize equipment that has the capability of producing more than a single product. For example, it may be beneficial to have the ability to utilize equipment typically used for the dehydrogenation of ethylbenzene to styrene also for the dehydrogenation of isoamylene to isoprene. It may be desirable to utilize commercial catalysts that are typically used in ethylbenzene to styrene reactions for the dehydrogenation of isoamylene to isoprene.

Efforts to utilize commercial catalysts that are typically used in ethylbenzene to styrene reactions for the dehydrogenation of isoamylene to isoprene have required high steam-to-hydrocarbon ratios and resulted in relatively short catalyst life. The higher steam-to-hydrocarbon ratio will increase the operating cost due to the need for more steam, therefore having an adverse effect on the economics of the process. Further, due to the decrease of catalyst activity, steaming of the catalyst is required in a regeneration step to restore activity. The operation of steaming the catalyst has a detrimental economic effect from the increased steam required and the reduction in product produced during this regeneration operation. The repeated action of steaming the catalyst typically results in a decrease in the useful life of the catalyst.

It may be desirable to be able to utilize equipment and catalysts typically used to dehydrogenate ethylbenzene to styrene also for the dehydrogenation of isoamylene to isoprene in a method that exhibits increased catalyst life with a reduction in the need for catalyst steaming.

### SUMMARY

Embodiments of the present invention generally include a method for producing isoprene by supplying a hydrocarbon feedstock containing isoamylene to a dehydrogenation reactor. The hydrocarbon feedstock and steam are contacted with a dehydrogenation catalyst within the reactor under conditions effective to dehydrogenate at least a portion of the isoamylene to produce isoprene. In an embodiment, the reactor is operated under a vacuum at a pressure of 1,000 mbar or less. A product is recovered from the dehydrogenation reactor containing isoprene.

The method can further include supplying steam to the dehydrogenation reactor in a steam to hydrocarbon molar ratio of at least 10:1 and operating the dehydrogenation reactor at a temperature of at least 300° C. The isoamylene to isoprene conversion can be at least 30%.

In an aspect, the dehydrogenation catalyst has an average effective pore diameter of at least 500 nanometers and has ferric oxide as a major component and potassium as a lesser component. In an embodiment, the dehydrogenation catalyst contains ferric oxide in amounts ranging from 40 wt % to 80 wt % and potassium oxide or potassium carbonate in an amount of about 5 wt % to 30 wt %.

The method can further include operating the dehydrogenation reactor at a steam to hydrocarbon molar ratio of at least 12:1, increasing the reactor temperature as needed to keep the isoamylene to isoprene conversion at least 35%, and where such catalyst deactivation during the isoamylene to isoprene dehydrogenation averages no more than 1° C. per day.

In another embodiment, the dehydrogenation reactor is operated at a steam to hydrocarbon molar ratio of at least 15:1 and at a pressure of 350 mbar or less. The reactor temperature is increased as needed to keep the isoamylene to isoprene conversion at least 40 wt %, and where such catalyst deactivation during the isoamylene to isoprene dehydrogenation averages no more than 0.5° C. per day.

In an embodiment the reactor and reactions are operable at least 30 days before the catalyst is a deactivated catalyst. In other embodiments the reactor and reactions are operable for at least 45 days, and alternatively at least or 60 days before the catalyst is a deactivated catalyst.

In an embodiment for a method of producing isoprene utilizing an ethylbenzene to styrene dehydrogenation reactor containing an EB dehydrogenation catalyst, a hydrocarbon feedstock containing isoamylene is supplied to the reactor along with steam in a steam to hydrocarbon molar ratio of at least 10:1. The hydrocarbon feedstock and steam are contacted with a dehydrogenation catalyst within the reactor at a pressure of 850 mbar or less and at a temperature of at least 500° C. The reactor temperature is increased as needed to keep a consistent isoprene content in the product such that the product contains an isoprene content equivalent to isoamylene to isoprene conversion of at least 30% and where such catalyst deactivation during the isoamylene to isoprene dehydrogenation averages no more than 1° C. per day.

Embodiments can operate at least to in excess of 30 days, 45 days, or 60 days before the catalyst is a deactivated catalyst.

Still another embodiment is for a method of producing isoprene in an ethylbenzene dehydrogenation reactor containing an EB dehydrogenation catalyst. The method includes modifying a dehydrogenation reactor to enable the removal of a vapor stream from the reactor and reduce the reactor pressure to vacuum conditions of 1,000 mbar or less and supplying a hydrocarbon feedstock containing isoamylene to the reactor and supplying steam to the dehydrogenation reactor in a steam to hydrocarbon molar ratio of at least 10:1. The hydrocarbon feedstock and steam are contacted with a dehydrogenation catalyst within the reactor which is operated at a temperature of at least 300° C. and vacuum conditions wherein substantially all of the hydrocarbons after the reactor are in a vapor phase. A vapor product containing isoprene is recovered from the dehydrogenation reactor.

The hydrocarbon feedstock can be at least 95 wt % isoamylene and the product can contain at least 30 wt % isoprene.

### BRIEF DESCRIPTION OF DRAWINGS

FIG. 1 illustrates results obtained from the dehydrogenation of isoamylene to isoprene at a steam to hydrocarbon molar ratio of 20:1 at pressures of 850 mbar and 340 mbar.

FIG. 2 illustrates results obtained from the dehydrogenation of isoamylene to isoprene at a steam to hydrocarbon molar ratio of 15:1 at a pressure of 340 mbar.

FIG. 3 illustrates results obtained from the dehydrogenation of isoamylene to isoprene at a steam to hydrocarbon molar ratios of 17:1 and 16:1 at pressures of 340 mbar and 290 mbar.

### DETAILED DESCRIPTION

The present invention involves the production of isoprene by dehydrogenating an isoamylene containing feed. The isoamylene feed is subjected to catalytic dehydrogenation under vacuum conditions that enable the dehydrogenation of the isoamylene to form a product having isoprene content equivalent to an isoamylene conversion of at least 30%. As used herein the phrase “conversion of at least xx %” means that at least xx weight percent of the isoamylene content in the feed converts to isoprene during the dehydrogenation process and is contained in the product stream.

In the dehydrogenation reaction, steam and isoamylene containing hydrocarbon feedstock can be supplied in a steam to hydrocarbon molar ratio of between 1:1 to about 25:1. The steam can be mixed with the hydrocarbon either prior to introduction to the reactor, or the steam and hydrocarbon can be supplied separately to the reactor through separate lines. The steam is condensed and forms a liquid portion, this liquid water along with any liquid hydrocarbons that may have been present in the feed or produced in the reaction, such as aromatics, for example benzene, toluene or xylene, can be drained from the reactor or a subsequent separation stage, in any suitable method. The reacted hydrocarbon can be removed as either a liquid or a vapor, depending on the reactor conditions.

Under the conditions of the present invention, substantially all of the produced isoprene and unreacted isoamylene containing feed are vaporized and are removed in a vapor phase by any suitable method, such as a vacuum compressor, which can maintain the reactor pressure at the desired vacuum conditions.

In an embodiment there are one or more reactors, in parallel or series, wherein the catalyst is located and one or more reaction zones exist. In addition to the reactor, there may be a subsequent separation stage that enables the liquid from the reactor to be recovered and the vapor product to be removed. A heat exchanger may also be utilized to cool the reaction effluent prior to the separation stage. The operating pressure of a separation stage may be essentially the same as the outlet pressure of the reactor, other than the pressure drop that may occur across the heat exchanger. In alternate embodiments the operating pressure of a separation stage may be different than the reactor. Methods and processes of dehydrogenation disclosed in U.S. patent application Ser. No. 11/811,084 filed Jun. 8, 2007 by Merrill, incorporated by reference herein, may be suitable for embodiments of the present invention.

The dehydrogenation catalyst can be any dehydrogenation catalyst having a large enough pore size in order to avoid excessive diffusion limitations leading to restriction of the conversion of isoamylene to isoprene, such as for a non-limiting example, those with an average effective pore diameter of at least 300 nanometers, at least 400 nanometers, or at least 500 nanometers. Subject to the pore diameter restrictions, the dehydrogenation catalyst may be of any suitable type, such as a catalyst containing iron as a major component with a lesser amount of potassium.

In a particular application of the invention the dehydrogenation catalyst is a ferric oxide, potassium carbonate based dehydrogenation catalyst having a relatively large average pore diameter, such as a pore diameter of at least 500 nanometers. Lesser amounts of cerium and other lanthanide group rare earths may also be present. Suitable catalyst compositions may comprise ferric oxide in amounts ranging from 40 to 80 wt %, potassium oxide or potassium carbonate in an amount of about 5 to 30 wt % and may also include lesser amount of cerium, and other suitable catalyst promoters. Catalysts disclosed in U.S. patent application Ser. No. 11/811,084 filed Jun. 8, 2007 by Merrill, incorporated by reference herein, may be utilized in the present invention.

In an aspect, the catalyst may be formed by mulling the iron and potassium components with, for example, a plastic hydraulic cement binder followed by extruding the material to form catalyst particles of about from 2.5 mm to 5.0 mm in diameter having an average effective pore diameter of at least 500 nanometers. More specifically the dehydrogenation catalyst may have an average effective pore diameter of at least 550 nanometers and may have an average effective pore diameter of between 550 nanometers and 2,000 nanometers.

The dehydrogenation catalyst can be, by non-limiting example: Styromax Plus from Sud-Chemie or Hypercat GV from Criterion.

In the present invention the LHSV can be any flow rate wherein the isoamylene to isoprene reaction can be achieved; such as for example embodiments of the invention can range from 0.1 hr⁻¹ to 10.0 hr⁻¹, or from 0.1 hr⁻¹ to 5.0 hr⁻¹.

During the experiments the steam-to-hydrocarbon molar ratio was varied from 20:1 to 16:1 and the reactor pressure was varied from 850 mbar to 290 mbar. Suitable steam-to-hydrocarbon molar ratio for embodiments of the invention can range from 10:1 to 30:1 or from 10:1 to 20:1. Suitable reactor pressure for the invention can range from 1000 mbar to 100 mbar and in particular embodiments can range from 900 mbar to 200 mbar.

Suitable reaction temperature for the invention can range from 300° C. to 800° C.

### EXPERIMENTAL EXAMPLES

Experiments were performed in which a dehydrogenation catalyst, such as those used for the conversion of ethylbenzene to styrene, was used for the reaction of isoamylene to isoprene. Catalysts that were tested are commercially available and included Styromax Plus from Sud-Chemie and Hypercat GV from Criterion. Other commercially available dehydrogenation catalysts were also tested with comparable results. The feed composition for the experiments was a mixture of about 93.8% 2-methyl-2-butene and about 6.2% 2-methyl-1-butene. In each of the experiments the flow rate of the input hydrocarbon stream was set at a LHSV of 0.35 hr⁻¹. During the various experimental runs the reaction temperature was adjusted at times in an effort to maintain a somewhat constant isoprene content in the product. The term “reaction temperature” as used herein refers to the reactor inlet temperature unless otherwise noted.

### Experiment 1

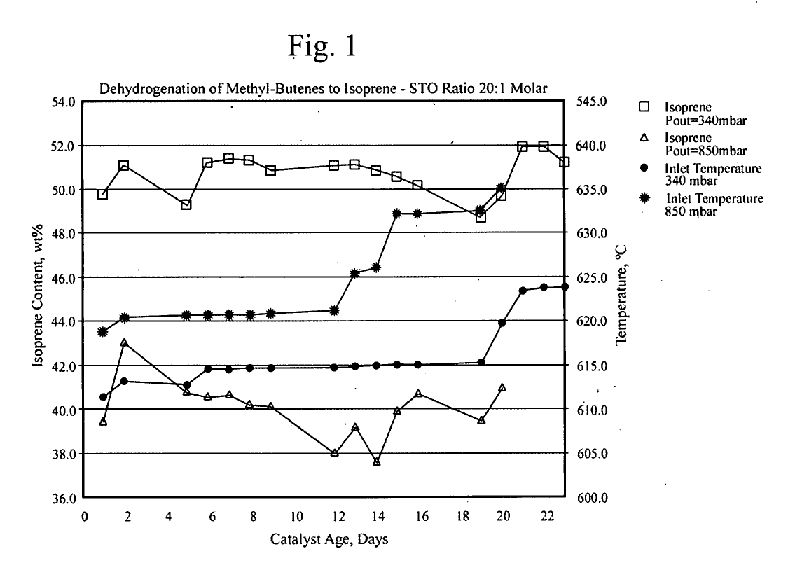

In Experiment #1 a steam-to-hydrocarbon molar ratio of 20:1 was used at a pressure of 850 mbar. The catalyst used was Styromax Plus from Sud-Chemie, which is a commercially available dehydrogenation catalyst. The results are shown in FIG. 1 wherein a product having an isoprene content of about 38 wt % to about 41 wt % was produced with a temperature starting at about 619° C. and rising to about 635° C. after 20 days. In the course of carrying out the dehydrogenation reaction, the catalyst becomes progressively deactivated resulting in a decrease in the isoprene content of the product and requiring the temperature to be steadily increased to maintain conversion. There was a cumulative increase of the reaction temperature of 15° C. that was observed over the 20 days of Experiment #1. The reactor temperature increase from catalyst deactivation averaged less than 1° C. per day over the isoamylene to isoprene dehydrogenation run.

The experimental results illustrate that a dehydrogenation catalyst that is typically used in the reaction of ethylbenzene to styrene can be used for the dehydrogenation of methylbutene to isoprene at a pressure of approximately 850 mbar or less, a steam-to-hydrocarbon molar ratio of at least 20:1, and can achieve sufficient conversion to produce a product having an isoprene content of at least 35 wt %. In this embodiment an average temperature increase of about 0.75° C. per day was observed over the isoamylene to isoprene dehydrogenation run.

The following table gives experimental data from Experiment 1.

| | Press | | | Isoprene |
|---|---|---|---|---|
| Day | mbar | Temp ° C. | SHR | wt % |
| 1 | 850 | 618.9 | 20 | 39.5 |
| 2 | 850 | 620.5 | 20 | 43 |
| 5 | 850 | 620.7 | 20.1 | 40.8 |
| 6 | 850 | 620.8 | 20 | 40.6 |
| 7 | 850 | 620.8 | 20 | 40.7 |
| 8 | 850 | 620.8 | 20 | 40.2 |
| 9 | 850 | 620.9 | 20 | 40.2 |
| 12 | 850 | 621.2 | 20 | 38 |
| 13 | 850 | 625.6 | 19.9 | 39.2 |
| 14 | 850 | 626.1 | 20 | 37.6 |
| 15 | 850 | 632.2 | 18.8 | 40 |
| 16 | 850 | 632.3 | 20 | 40.7 |
| 19 | 850 | 632.7 | 20.1 | 39.5 |
| 20 | 850 | 635.3 | 20 | 41 |
| 21 | 850 | 626.4 | 14.4 | 32.8 |
| 22 | 850 | 640.9 | 14.3 | 34 |
| 26 | 850 | 625.5 | 17.3 | 27.8 |
| 27 | 850 | 631.1 | 17.3 | 39.1 |
| 28 | 850 | 630.8 | 17.3 | 45.5 |
| 31 | 850 | 632.5 | 17.4 | 37.1 |
| 32 | 850 | 639.1 | 17.3 | 56.5 |
| 33 | 850 | 629.8 | 17.4 | 41.1 |
| 34 | 850 | 636.7 | 17.4 | 35.7 |
| 35 | 849.9 | 630.1 | 17.3 | 40.4 |
| 39 | 850 | 640.5 | 17.4 | 35.8 |
| 40 | 850 | 630.6 | 17.4 | 34.1 |
| 41 | 850 | 631.7 | 17.4 | 35.7 |
| 42 | 850 | 638 | 19.4 | 40.8 |
| 43 | 850 | 638.2 | 19.4 | 40 |
| 44 | 850 | 638.1 | 19.5 | 38.3 |
| 47 | 850 | 638.6 | 19.4 | 37.5 |

### Experiment #2

In Experiment #2 operating conditions of a steam-to-hydrocarbon molar ratio of 20:1 was used at a pressure of 340 mbar. The catalyst used was Sud-Chemie Styromax Plus. The results are shown in FIG. 1 wherein a product having an isoprene content of about 49 wt % to about 52 wt % was produced with a temperature starting at about 611° C. and rising to about 624° C. after 23 days. In the course of carrying out the dehydrogenation reaction, the catalyst showed some deactivation requiring the temperature to be increased on days 20 and 21 to maintain isoprene content above 50 wt %. A total of a 13° C. increase in the reaction temperature was observed over the 23 days of Experiment #2.

The experimental results shown in FIG. 1 illustrate that reducing the pressure results in an increase in conversion at a consistent steam-to-hydrocarbon molar ratio, in Experiment #2 pressure reduction from approximately 850 mbar to approximately 340 mbar can result in an increase in conversion to produce a product having an increased isoprene content, in this case of at least 45 wt % at the same steam-to-hydrocarbon molar ratio of 20:1. This increase in conversion is found to occur at a reduced temperature. In this embodiment an average temperature increase from catalyst deactivation of about 0.5° C. per day was observed over the isoamylene to isoprene dehydrogenation run.

### Experiment #3

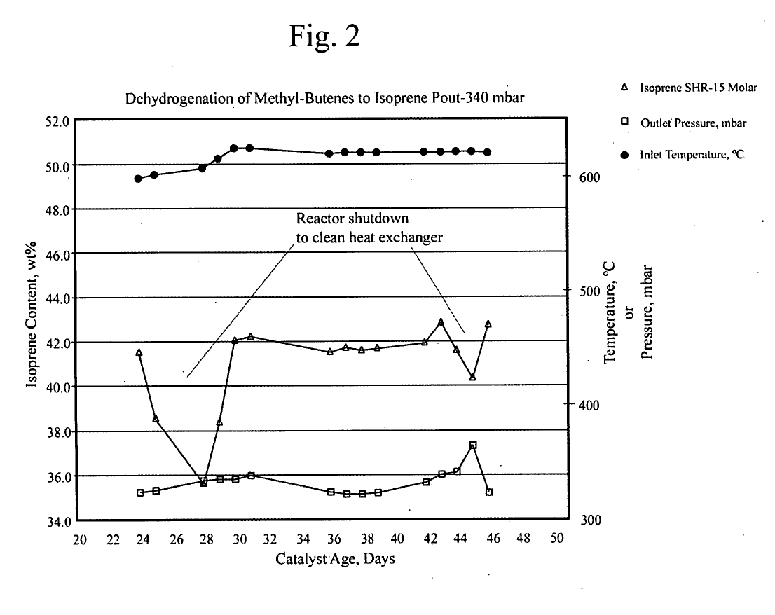

In Experiment #3 the reaction from Experiment #2 was continued although with a reduced steam-to-hydrocarbon molar ratio of 15:1 at the pressure of approximately 340 mbar. The results are shown in FIG. 2 wherein a product having an isoprene content of about 36 wt % to about 43 wt % was produced with a reaction temperature starting at about 600° C. on day 24 and ending at about 622° C. on day 46 for an overall 22° C. increase over the 22 day run. An initial temperature rise of 26° C. occurred in the first seven days, which then decreased to a temperature rise of about 22° C. and stabilized at a reaction temperature of about 622° C. over the remaining 15 days of Experiment #3.

Reducing the steam-to-hydrocarbon molar ratio from 20:1 to 15:1 resulted in a corresponding reduction in the isoprene content of the product, in these experiments from about 50 wt % to about 42 wt %. In Experiment #3 an average temperature increase of about 1.0° C. per day was observed over the experimental run, although no significant temperature rise was observed during the final ten days of the experimental run, indicating a steady-state operation without significant catalyst deactivation.

### Experiment #4

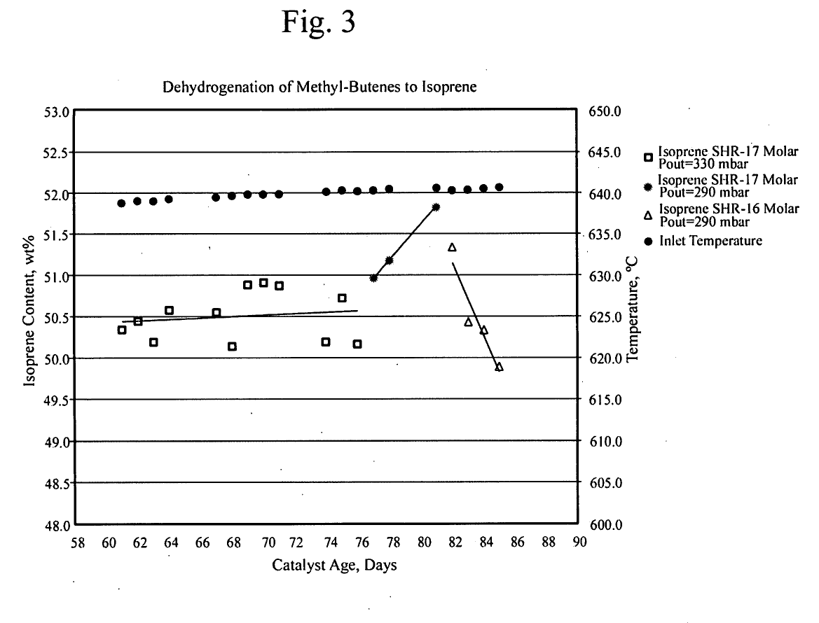

In Experiment #4 the reaction from Experiments #2 and #3 was continued although with an increased steam-to-hydrocarbon molar ratio of 17:1 at a pressure of approximately 330 mbar. The results are shown in FIG. 3 wherein a product having an isoprene content of about 50 wt % to about 51 wt % was produced with a temperature starting at about 639° C. on day 61 and rising to about 641° C. on day 76. A 2° C. increase was observed over the 15 days of Experiment #4. Experiment #4 showed a stable reaction at a steam-to-hydrocarbon molar ratio of 17:1 and a pressure of 330 mbar while producing a product of slightly more than 50 wt % isoprene.

Increasing the steam-to-hydrocarbon molar ratio from 15:1 to 17:1 resulted in maintaining a stable reaction producing a product having an increased isoprene content of approximately 50 wt %. In this experiment an average temperature increase from catalyst deactivation of less than 0.2° C. per day was observed. The slight rise in temperature can also be seen to correspond with a slight increase of isoprene content in the product, indicating steady-state operation with stable conversion without significant catalyst deactivation.

### Experiment #5

In Experiment #5 the experimental conditions and reaction of Experiment #4 was continued with a steam-to-hydrocarbon molar ratio of 17:1, except that the pressure was reduced to 290 mbar. The results are shown in FIG. 3 wherein the isoprene content increased to 51 wt % on day 77 and further increased to almost 52 wt % on day 81 while the temperature increased by only 0.1° C. over these days. Experiment #5 illustrates the benefit of reduced reaction pressure on the conversion of methylbutenes to isoprene to produce a product with increased isoprene content without significant catalyst deactivation. This experiment demonstrated a stable reaction at a steam-to-hydrocarbon molar ratio of 17:1 and a pressure of 290 mbar while producing a product of greater than 50 wt % isoprene.

Reducing the pressure to approximately 290 mbar while maintaining a steam-to-hydrocarbon molar ratio of 17:1 resulted in maintaining a stable reaction of product having an isoprene content of more than 50 wt %. In this embodiment an average temperature increase from catalyst deactivation of about 0.025° C. per day was observed.

### Experiment #6

In Experiment #6 the reaction of Experiment #5 was continued with a pressure of 290 mbar although the steam-to-hydrocarbon molar ratio was reduced to 16:1. The results are shown in FIG. 3 wherein the isoprene content showed a decreasing trend from 51.4 wt % on day 82 to about 49.9 wt % on day 85 while the temperature remained steady. Experiment #6 illustrates that a reduction in steam-to-hydrocarbon molar ratio from 17:1 to 16:1 and constant pressure will result in decreasing isoprene content, which was expected.

Reducing the steam-to-hydrocarbon molar ratio to 16:1 at the steady pressure of approximately 290 mbar exhibited a decreasing trend in the isoprene content in the product of approximately 0.5 wt % per day during the four days of the experiment. The decrease in conversion may possibly have been overcome by an increase in the reactor temperature. It is also possible that the isoprene content may have stabilized at a level between 40 wt % and 50 wt % if the reaction had been allowed to continue. In experiment #3 with a steam-to-hydrocarbon molar ratio of 15:1 at 340 mbar the isoprene content in the product was about 42 wt %; therefore at a steam-to-hydrocarbon molar ratio of 16:1 at 290 mbar the isoprene content in the product would be expected to be greater than 42 wt %. Also of note is that at this point in the experiments the catalyst had been in continuous service for over 80 days and had been subjected to the many changing conditions described above, thereby possibly having some lack of activity and selectivity due to the extended run.

The following table gives experimental data from Experiments 2 through 6.

| | Press | | | Isoprene |
|---|---|---|---|---|
| Day | mbar | Temp ° C. | SHR | wt % |
| 1 | | 611.4 | 20 | 49.8 |
| 2 | 343 | 613.1 | 20 | 51.1 |
| 5 | 431 | 612.7 | 20 | 49.3 |
| 6 | 335 | 614.6 | 20 | 51.2 |
| 7 | 335 | 614.5 | 20 | 51.4 |
| 8 | 335 | 614.7 | 20 | 51.4 |
| 9 | 335 | 614.7 | 20 | 50.9 |
| 12 | 337 | 614.7 | 20 | 51.1 |
| 13 | 339 | 614.9 | 20 | 51.2 |
| 14 | 337 | 614.9 | 20 | 50.9 |
| 15 | 338 | 615 | 20 | 50.6 |
| 16 | 338 | 615 | 20 | 50.2 |
| 19 | 346 | 615.3 | 20 | 48.7 |
| 20 | 355 | 619.8 | 20 | 49.7 |
| 21 | 355 | 623.5 | 20 | 51.9 |
| 22 | 363 | 623.8 | 20 | 51.9 |
| 23 | 369 | 623.9 | 20 | 51.2 |
| 24 | 324 | 599.2 | 20 | 41.5 |
| 25 | 325 | 602.6 | 15 | 38.6 |
| 28 | 334 | 607.8 | 15 | 35.6 |
| 29 | 335 | 616.7 | 15 | 38.4 |
| 30 | 335 | 625.6 | 15 | 42.1 |
| 31 | 338 | 626 | 15 | 42.3 |
| 36 | 324 | 621.1 | 15 | 41.6 |
| 37 | 322 | 621.7 | 15 | 41.7 |
| 38 | 323 | 621.8 | 15 | 41.6 |
| 39 | 323 | 621.9 | 15 | 41.7 |
| 42 | 332 | 622 | 15 | 41.9 |
| 43 | 339 | 622.2 | 15 | 42.9 |
| 44 | 341 | 622.3 | 15 | 41.6 |
| 45 | 365 | 622.4 | 15 | 40.4 |
| 46 | 323 | 621.6 | 15 | 42.8 |
| 49 | 336 | 626.5 | 20 | 51.1 |
| 50 | 330.3 | 627 | 20 | 50.7 |
| 53 | 357.3 | 630.1 | 20 | 50.8 |
| 54 | 339.4 | 625.3 | 20.3 | 50.7 |
| 55 | 343.8 | 625.4 | 20.3 | 50.3 |
| 56 | 338.3 | 631 | 17 | 49.4 |
| 57 | 354.7 | 634.5 | 17 | 49.7 |
| 60 | 350.4 | 638.7 | 17 | 50.7 |
| 61 | 353 | 638.8 | 17 | 50.3 |
| 62 | 330.6 | 639 | 17 | 50.4 |
| 63 | 341.4 | 639.1 | 17 | 50.2 |
| 64 | 329.2 | 639.3 | 17 | 50.6 |
| 67 | 326.9 | 639.5 | 17 | 50.5 |
| 68 | 344.2 | 639.7 | 17 | 50.2 |
| 69 | 321.1 | 639.8 | 17 | 50.9 |
| 70 | 328.2 | 639.9 | 17 | 50.9 |
| 71 | 317.4 | 639.9 | 17 | 50.9 |
| 74 | 349.4 | 640.2 | 17 | 50.2 |
| 75 | 347.1 | 640.3 | 17 | 50.7 |
| 76 | 334.1 | 640.3 | 17 | 50.2 |
| 77 | 296 | 640.4 | 17 | 51 |
| 78 | 273.1 | 640.5 | 17 | 51.2 |
| 81 | 297.2 | 640.7 | 17 | 51.8 |
| 82 | 292.1 | 640.4 | 16 | 51.3 |
| 83 | 292.1 | 640.5 | 16 | 50.4 |
| 84 | 285.2 | 640.6 | 16 | 50.3 |
| 85 | 283.1 | 640.7 | 16 | 49.9 |

Various terms are used herein, to the extent a term used in not defined herein, it should be given the broadest definition persons in the pertinent art have given that term as reflected in printed publications and issued patents.

The term “activity” refers to the weight of product produced per weight of the catalyst used in a process per hour of reaction at a standard set of conditions (e.g., grams product/gram catalyst/hr).

The term “conversion” refers to the weight percent of a component in the feed that converts to a new component in the product stream during the dehydrogenation process.

The term “deactivated catalyst” refers to a catalyst that has lost enough catalyst activity to no longer be efficient in a specified process. Such efficiency is determined by individual process parameters.

The term “EB dehydrogenation catalyst” refers to a catalyst that has the capability to catalyze the dehydrogenation reaction of ethylbenzene to styrene. The EB dehydrogenation catalyst is not limited to a commercially available catalyst or one that is commercial use for the dehydrogenation of ethylbenzene to styrene. The term EB dehydrogenation catalyst but would include those catalysts that are in commercial use for the dehydrogenation reaction of ethylbenzene to styrene and catalysts that are commercially available for the dehydrogenation reaction of ethylbenzene to styrene.

The term “isoamylene” as used herein is meant to refer to any methylbutene in one or more of the isomeric states and in combinations thereof. The methylbutene isomers are: 2-methyl-2-butene; 2-methyl-1-butene; and 3-methyl-1-butene. Isoprene can be produced by the catalytic dehydrogenation reaction of methylbutene isomer 2-methyl-2-butene in the presence of oxygen. Upon the reaction of the 2-methyl-2-butene isomer to isoprene, the equilibrium between the isomers will be disrupted and can result in the formation of additional 2-methyl-2-butene from the other two isomers, so although the reactant to form isoprene may be the 2-methyl-2-butene isomer, it is possible for the other isomers to convert to 2-methyl-2-butene via an isomerization reaction and thereby become a reactant to form isoprene.

The term “regenerated catalyst” refers to a catalyst that has regained enough activity to be efficient in a specified process. Such efficiency is determined by individual process parameters.

The term “regeneration” refers to a process for renewing catalyst activity and/or making a catalyst reusable after its activity has reached an unacceptable/inefficient level. Examples of such regeneration may include passing steam over a catalyst bed or burning off carbon residue, for example.

Depending upon the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Other and further embodiments, versions and examples of the invention may be devised without departing from the basic scope thereof and the scope thereof is determined by the claims that follow.

What is claimed is:

1. A method for the production of isoprene comprising:
contacting a hydrocarbon feedstock containing isoamylene with a dehydrogenation catalyst, at a pressure of 1,000 mbar or less, under reaction conditions effective to dehydrogenate at least a portion of said isoamylene to produce isoprene.

2. The method of claim 1, further comprising:
supplying steam to the dehydrogenation reaction in a steam to hydrocarbon molar ratio of at least 10:1; and
operating the dehydrogenation reaction in a reactor at a temperature of at least 300° C.

3. The method of claim 1, wherein the isoamylene to isoprene conversion is at least 30%.

4. The method of claim 1, wherein the dehydrogenation catalyst has an average effective pore diameter of at least 500 nanometers.

5. The method of claim 1, wherein the dehydrogenation catalyst has ferric oxide as a major component and potassium as a lesser component.

6. The method of claim 1, wherein the dehydrogenation catalyst contains ferric oxide in amounts ranging from 40 wt % to 80 wt %, and potassium oxide or potassium carbonate in an amount of from about 5 wt % to 30 wt %.

7. The method of claim 2, further comprising:
operating the dehydrogenation reaction at a steam to hydrocarbon molar ratio of at least 12:1;
increasing a temperature of the reactor as needed to keep the isoamylene to isoprene conversion at least 35%; and
wherein the reactor temperature increase from catalyst deactivation averages no more than 1° C. per day.

8. The method of claim 2, further comprising:
operating the dehydrogenation reactor at a steam to hydrocarbon molar ratio of at least 12:1;
operating the dehydrogenation reactor at a pressure of 850 mbar or less;
increasing the reactor temperature as needed to keep the isoamylene to isoprene conversion at least 40%; and
wherein the reactor temperature increase from catalyst deactivation averages no more than 1° C. per day.

9. The method of claim 2, further comprising:
operating the dehydrogenation reactor at a steam to hydrocarbon molar ratio of at least 14:1;
operating the dehydrogenation reactor at a pressure of 400 mbar or less;
increasing the reactor temperature as needed to keep the isoamylene to isoprene conversion at least 40%; and
wherein the reactor temperature increase from catalyst deactivation averages no more than 1° C. per day.

10. The method of claim 2, further comprising:
operating the dehydrogenation reactor at a steam to hydrocarbon molar ratio of at least 12:1;
operating the dehydrogenation reactor at a pressure of 350 mbar or less;
increasing the reactor temperature as needed to keep the isoamylene to isoprene conversion at least 40%; and
wherein the reactor temperature increase from catalyst deactivation averages no more than 1° C. per day.

11. The method of claim 2, further comprising:
operating the dehydrogenation reactor at a steam to hydrocarbon molar ratio of at least 15:1;
operating the dehydrogenation reactor at a pressure of 350 mbar or less;
increasing the reactor temperature as needed to keep the isoamylene to isoprene conversion at least 40%; and
wherein the reactor temperature increase from catalyst deactivation averages no more than 0.5° C. per day.

12. The method of claim 1, wherein the reaction can operate in excess of 30 days before the catalyst is a deactivated catalyst.

13. The method of claim 1, wherein the reaction can operate in excess of 45 days before the catalyst is a deactivated catalyst.

14. The method of claim 1, wherein the reaction can operate in excess of 60 days before the catalyst is a deactivated catalyst.

15. A method for the production of isoprene utilizing an ethylbenzene to styrene dehydrogenation reactor containing an ethylbenzene to styrene dehydrogenation catalyst comprising:
supplying a hydrocarbon feedstock comprising isoamylene to a dehydrogenation reactor;
supplying steam to the dehydrogenation reactor in a steam to hydrocarbon molar ratio of at least 10:1;
contacting the hydrocarbon feedstock and steam with a dehydrogenation catalyst within a reaction zone;
operating the dehydrogenation reactor at a pressure of 850 mbar or less;
operating the dehydrogenation reactor at a temperature of at least 500° C. and increasing the reactor temperature as needed to keep a consistent isoprene content in the product;
recovering a product from the dehydrogenation reactor containing an isoprene content equivalent to isoamylene to isoprene conversion of at least 30%; and
wherein the rate of temperature increase of the reactor from catalyst deactivation averages no more than 1° C. per day.

16. The method of claim 15, further comprising:
operating the dehydrogenation reactor at a steam to hydrocarbon molar ratio of at least 12:1;
operating the dehydrogenation reactor at a pressure of 500 mbar or less; and
increasing the reactor temperature as needed to keep the isoamylene to isoprene conversion at least 40%.

17. The method of claim 15, further comprising:
operating the dehydrogenation reactor at a steam to hydrocarbon molar ratio of at least 12:1;
operating the dehydrogenation reactor at a pressure of 350 mbar or less;
increasing the reactor temperature as needed to keep the isoamylene to isoprene conversion at least 45%.

18. The method of claim 15, further comprising:
operating the dehydrogenation reactor at a steam to hydrocarbon molar ratio of at least 15:1;
operating the dehydrogenation reactor at a pressure of 300 mbar or less;
increasing the reactor temperature as needed to keep the isoamylene to isoprene conversion at least 50%; and
wherein the reactor temperature increase from catalyst deactivation averages no more than 0.5° C. per day.

19. The method of claim 15, wherein the reactor is operated for at least 30 days before the catalyst is a deactivated catalyst.

20. The method of claim 15, wherein the reactor is operated for at least 45 days before the catalyst is a deactivated catalyst.

21. The method of claim 15, wherein the reactor can operate in excess of 60 days before the catalyst is a deactivated catalyst.

22. A method for the production of isoprene in an ethylbenzene dehydrogenation reactor containing a catalyst for ethylbenzene to styrene dehydrogenation comprising:
modifying the dehydrogenation reactor to enable the removal of a vapor stream from the reactor and reduce the reactor pressure to vacuum conditions of 1,000 mbar or less;
supplying a hydrocarbon feedstock comprising isoamylene to the reactor;
supplying steam to the dehydrogenation reactor in a steam to hydrocarbon molar ratio of at least 10:1;
contacting the hydrocarbon feedstock and steam with a dehydrogenation catalyst within a reaction zone;
operating the dehydrogenation reactor at a temperature of at least 300° C. and vacuum conditions wherein substantially all of the hydrocarbons are in a vapor phase; and
recovering a vapor product from the dehydrogenation reactor comprising isoprene.

23. The method of claim 22, wherein the hydrocarbon feedstock is comprised of at least 95 wt % isoamylene, and wherein the product is comprised of at least 30 wt % isoprene.
